(19) United States
(12) Patent Application Publication
(10) Pub. No.: US 2023/0240575 A1
(43) Pub. Date: Aug. 3, 2023

(54) SYSTEM AND METHOD OF PSYCHOMETRIC TEST & ANALYSIS FOR ASSESSING DIGITAL QUOTIENT

(71) Applicant: Rahul SHANDILYA, Noida (IN)

(72) Inventor: Rahul SHANDILYA, Noida (IN)

(21) Appl. No.: 18/101,392

(22) Filed: Jan. 25, 2023

(30) Foreign Application Priority Data
Jan. 25, 2022 (IN) 202211004323

(51) Int. Cl. A61B 5/16; G06F 16/35

(52) U.S. Cl. CPC A61B 5/167; G06F 16/353

(57) ABSTRACT

The invention is directed to a system and method for using psychometric testing & analysis based on a framework of individual's personality/behavioral traits which helps assess the digital quotient of an individual or an organization.

### FIELD OF INVENTION

The invention is directed to a system and method for using psychometric testing & analysis based on a framework of individual's personality/behavioral traits which helps assess the digital quotient of an individual or an organization.

### BACKGROUND OF INVENTION

In existing systems or methods known for psychometric testing and analysis towards assessment of Digital Quotient

### BRIEF DESCRIPTION OF DRAWINGS

FIG. 1 illustrates the systematic method for using the framework of personality traits and Psychometric Testing & Analysis for assessment of digital quotient.

FIG. 2 illustrates the spider map of seven traits and associated score of assessment

### OBJECT OF THE INVENTION

The main object of the invention is to define method using the personality traits (termed as an acronym—UCCCEEE framework) towards assessment of digital quotient of individuals and organizations and represent the outcome in two forms as DQME SCORE and DQME TYPE INDICATORS.

The second object of the invention is to provide individuals to leverage “Assessment of their Digital Quotient Profiling” using the above given method and take appropriate interventions to improve upon their Digital Quotient.

The third object of the invention is to provide organizations to conduct assessment of their existing employees or potential new hires and plan appropriate interventions for improving their DQ while preparing for the journey of the digital transformation

The fourth object of the invention is to provide organizations to assess Digital Quotient of the individuals during the organizational talent management process and deploy suitable interventions during talent development and growth process.

The fifth object of the invention is to provide state governance, corporates and societies at large to build human resources and societal characters towards the digital economy respectively and leverage them towards various policy development and deployments.

### DETAILED DESCRIPTION OF THE INVENTION

The invention is directed towards a system and method of to assess digital quotient of an organization or individual based on psychometric test conducted by using personality traits of wherein the traits are identified based on the influence they make on the digital quotient of the individual.

According to the inventor of the invention the digital quotient for an individual is described as:

“The measure of awareness, responsiveness, and adoption of emerging digital technologies is commonly referred to as the individual person's digital quotient. It helps in measuring the correlation between an individual person and the digital ecosystem.”

According to the inventor there are various drivers (Personality Traits) of an individual person which influence th digital quotient which eventually reflects the individual's behavior over/towards the digital platforms/technologies.

Among 45 plus behaviors which may have some influence on an individual's response & adoption over a digital platform or medium—seven (07) personality traits were identified which make significant influence and are derivative of all the 45 plus behaviors. These seven traits are identified on the criteria of effectiveness, consistency and dominance and can define and predict how an individual person would behave should there be any stimulus sent to him/her over any digital medium. These traits are—Updated, Confident, Connected, Curious, Efficient, Epicurean and Exprimentative.

The deductive analysis leveraged the natural language processing (NLP) methods, word cloud models, relevance scores and technologies on text mining and keyword extraction.

The individual in this method may be considered as a person or as a legal entity like an organization. The applicant researcher also concluded that these seven personality drivers based UCCCEEE framework can also represent on collective basis example—a team or an organization and therefore can be applied to their collective behaviors as well.

The traits have been organized in the sequence (UCCCEEE) for the better recall purposes and do not change the purpose and cause, even if they would be organized in any other permutation or manner or form or syntax.

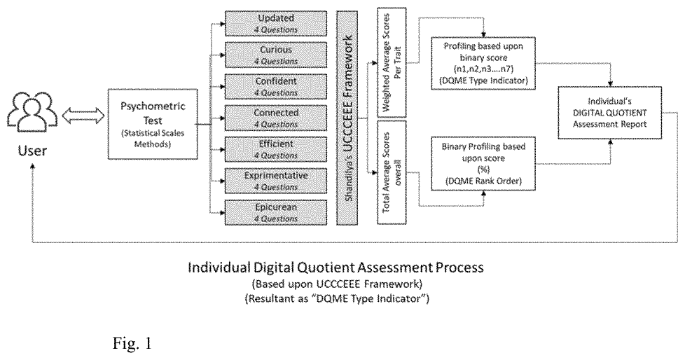

FIG. 1.0: UCCCEEE FRAMEWORK Based DQ Assessment Process

07 Personality & Behavioral Traits Reflecting the Digital Quotient and their Description

Psychometric Test and Rating Process Leading into 02 Score Cards—Submitted for Patent Registration

The assessment process of an individual person is based upon psychometric test of having situational questions per trait which an individual is expected to respond as naturally as possible.

These questions are presented to a respondent under 7 categories each one based upon 7 personality traits of UCCCEEE framework.
1. UPDATED,
2. CONFIDENT,
3. CONNECTED,
4. CURIOUS,
5. EFFICIENT,
6. EPICUREAN,
7. EXPERIMENTATIVE.

These “seven” categories of personality traits cover over 45 different behaviors which are factored in arriving at this framework. Thus, the psychometric test method using the UCCCEEE framework invariably covers these sub-traits while considering the digital quotient assessment of the target respondent/individual or an entity like an organization, society etc. These 45 different behaviors are as given below
Ease of Usage (EOU), The Fear of Missing Out (FOMO), The Fear of Change (FOC), Expression of having a desire for an Alternate Profile, Liking for efficiency in lifestyle, expression of confidence, Seek out Popularity among the surrounding ecosystem and look for self-differentiation, Representing through a Virtual Avatar beyond physical boundaries, Being Experimentative, Reflecting Age based behaviors—faster learning curve, Deeper liking for Personalization—choices driven out of personal likes and dislikes r, Self-Gratification/Entertainment—seeking diversified/personalized content, Staying Current/Updated, Aligning with Trends, Seeking Happiness, Staying Connected with social ecosystem, Enhanced liking for the Socialization, Driving through Networking, Having extra insistence of the privacy and Security, Display linguistic barriers, Economic Affordability, Seeking reasonable impact, Anxious to hear News, Creative and Innovating approach, Continuous Learning, Making significant Influence, Staying always Relevant, Deep Expressions, Gathering and Retention of Knowledge, Logical Reasoning, Openness and Frankness, Impact of Gender differentiation, Quality of Education, Scale and time of Chatting, Securing Privacy, Playful and Adventurous, Nerdy and Technology driven, Desire to display degree of awareness, Economic disparity through the family & individual Income, Reach and accessibility, Product Offerings and Features, Physical touch feel, Outright Materialistic and Ambitious, Seeking Convenience and Economics Models & offers.

Each category has minimum 2 or more questions presenting situations and seek responses from the individual undertaking the test. The questions are organized as direct correlation or indirect correlational. The questions can vary based upon the target respondent group—example corporate, home makers, academics, children, direct consumer base etc. and not just limited to given here.

According to the inventor the no and type of questions in the psychometric test is subject to change to keep alignment with the evolving ecosystem.

The responses are on the “Range Scale” of 1 to 100 and respondent can score anywhere in between this range. There are two score cards for an individual person as drawn out of the responses. These are as below

Score Card 1: DQME Rank Order Score

This DQME Rank Order Score is a direct sum-total measure of respondent's digital quotient. It represents a collective value of response to the situational questions under all 7 trait categories in the psychometric test paper which is to be completed in one seating by the respondent. The score is on a range scale of 1 to 100. The higher the score, more it is conducive to the positive response to a stimulus at a very high level. It can be seen as high-high positive correlation.

Score Card 2: DQME Type Indicator Score

This patent application propagates that DQ (Digital Quotient) of an individual is influenced by 7 different traits which play key role in defining your response to any stimulus sent to you over a digital medium.

They are covered as UCCCEEE framework with range score as given where “0” means least responsive and “100” means most responsive. Th point of inflexion means where the behavior turns to the other side.
1. UPDATED (between 0 to 100 and point of inflexion as 50)
2. CONFIDENT (between 0 to 100 and point of inflexion as 50)
3. CONNECTED (between 0 to 100 and point of inflexion as 50)
4. CURIOUS (between 0 to 100 and point of inflexion as 50)
5. EFFICIENT (between 0 to 100 and point of inflexion as 50)
6. EPICUREAN (between 0 to 100 and point of inflexion as 50)
7. EXPERIMENTATIVE (between 0 to 100 and point of inflexion as 50)

The respondent answers situational questions per category under each of the 07 trait categories and weighted average score per trait categories are calculated on a range score of 1 to 100.

For the purpose of representation in binary mode—the weighted average score between 0 to 50 is represented as “0” and score between 51 to 100 is considered as “1”.

The representation of the DQME Type Indicator Score is considered as sequence of “Binary Scores” across the 07 traits in the sequence as given above.
a. Example 1: A individual can get a score represented as 1010101.
b. Example 2: An individual who has very high digital quotient should have type indicator of 1111111
c. Example 3: An individual who has very low digital quotient should have type indicator of 0000000

There could many permutations of the scores and can be represented in the format as above but with score varying on the individual traits.

The score for example 1 may appear like this

DQME Type Indicator Score: 0101010

In the above example the weighted average score for each of the trait's category on the psychometric test paper would fall between the ranges as below:

Range of
BINARY RANGE CODE FOR WEIGHTED AVERAGE SCORE FOR UPDATED: 0-50;
BINARY CODE FOR WEIGHTED AVERAGE SCORE FOR CONFIDENT: 50-100;
BINARY CODE FOR WEIGHTED AVERAGE SCORE FOR CONNECTED: 0-50;
BINARY CODE FOR WEIGHTED AVERAGE SCORE FOR CURIOUS: 50-100;
BINARY CODE FOR WEIGHTED AVERAGE SCORE FOR EFFICIENT: 0-50;
BINARY CODE FOR WEIGHTED AVERAGE SCORE FOR EPICUREAN: 50-100;
BINARY CODE FOR WEIGHTED AVERAGE SCORE FOR EXPERIMENTATIVE: 0-50

The higher is the weighted average score on these individual traits for a respondent, there shall be more positive responsive strokes generated by the respondent to any stimulus sent on the digital media.

However, the overall indication of response of an individual is best represented through a collective and correlational representation of the binary code (s) of an individual (within that a specific score will give a certainly closer assessment of the behavior). Thus, a careful combination of the study of the individual traits shall provide more useful reference of the DQME TYPE SCORE.

Visual Representation of the DQME Type Indicator Score

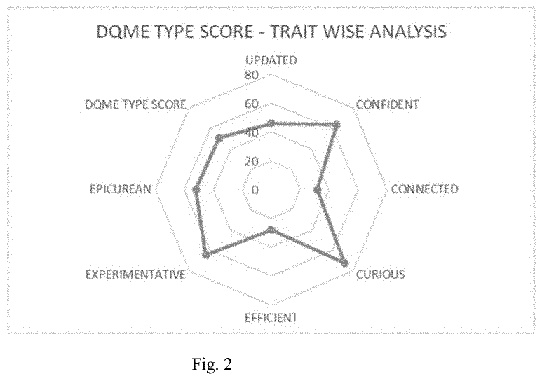

A standard visual representation of DQME Type Indicator score is in the format of a spider chart FIG. 2) and is associated with commentary based on each trait score and its influence, correlation between each trait score and a collective influence and overall response mechanism of an individual.

The commentary is proposed to be termed as Digital Quotient Profiling of an Individual/Entity.

FIG. 2.0 Sample Chart: Spider Map of the 7 Traits Based DQME Score of an Individual

The psychometric test is also automated using software programming tools (web3.0 framework) and shall continue to get enhanced with the evolution of the technologies.

The data collection for the test is done over online applications, wherein, the design provides for the respondent to drag and drop the desired value over the range scale OR state a specific value in the given box.

An advanced version of the test shall provide an alternative to the online applications by leveraging voice Interface and artificial intelligence tools for conducting voice interview with the respondent across situational questions and then using NLP algorithms to derive at the weighted average score for the given trait.

The calculation method for both scores as stated above and representation do not change even if the input mechanism may vary depending upon the technology usage varying over time.

Use of the Natural Language Processing (Artificial Intelligence) in Identifying the UCCCEEE Framework

The following statistical methods were used in identifying the “seven” traits
1. Content Word cloud—word frequency cluster wise and using frequency as a differentiator selecting most frequently preferred content words by respondents while expressing behavior towards digital technologies adoption
2. Relevance score for words: calculated based collocation in the text responses—relative rank ordering of these content words on conjugal appearances one or more times.
   RELEVANCE SCORE: Function (RS)=(β/α)(1),
      where RS: relevance score
      α=independent term frequency
      β=associated term frequency (all combinations where the keyword was collocated with other keywords and in text responses)
      μ=α+β=Total content words collected in the bag of words for the cluster.
      Relevance score method gives the moderation towards identifying the uniqueness of the above said content word. This enables identification of the priority ranking of the content words and wherever more than one cluster were involved, the RS were considered as aggregated values for many clusters.
3. NLP based text mining and analytics tools.
   a. Term Frequency: TF
      Term frequency (TF) suggests how many times (frequently) a word occurs in all the documents as a ratio with all the words in the document.

$${TF}_{i , j} = \frac{{n}_{i , j}}{{\sum }_{k} {n}_{i , j}}$$

FIG. 3.0
where n=number of times the word has occurred and is divided by the number of terms in a document. Note: We considered 38 words identified in Section 2.
b. Inverse Document Frequency: IDF
   Inverse document frequency (IDF) indicates the chances of occurrence of a term across multiple documents. It is calculated as the logarithm of the ratio of the number of documents in the survey divided by the number of documents where the specific term appears.

$${idf}_{j} = log [ \frac{n}{{df}_{i}} ]$$

FIG. 4.0
It is calculated as (3) where n represents the total number of documents and is divided by the number of documents containing the given term.
c. TF-IDF: Term Frequency and Inverse Document Frequency Relationship
   The TF-IDF methodology was used to extract keywords that are most frequently used by respondents across all clusters. γ (TF-IDF) is the multiplication of the above two resultants. It is calculated as follows:

$${w}_{i , j} = {tf}_{i , j} \times log ( \frac{n}{{df}_{i}} )$$

tfᵢⱼ=number of occurrences of i in j
dfᵢ=number of documents containing i
N=total number of documents

Applying the Same Method for an Entity Instead of an Individual

The case of an entity like an organization—the score is considered as weighted average of individual score of all the participants from the organization as selected for participating in the test. The psychometric test administered should be on Cronbach Alpha score of 0.8 or above and sample to selected based on stratified random sampling method.

The DQME Profile of an organization (entity) is considered based on the weighted average DQME Type Indicator score of each trait across participating representative individuals. The DQME Rank Order for an organization (entity) is considered as weighted average score of all individuals who participated based on stratified random sampling as above.

Table Signifying the Explanation of the 07 Traits in UCCCEEE Framework

TABLE 1.0
UCCCEEE framework and seven personality & behavioral traits

| SI. No | Attribute Type | Description |
|---|---|---|
| 1 | Updated | Has a liking for gathering data & information, often demonstrates eye for |
| | | details, and feels comfortable in inflow of the information, even finds |
| | | information overload an acceptable practice. |
| 2 | Curious | Displays high level of eagerness towards seeking better understanding, in |
| | | depth knowledge, operates proactively and reaches out to different sources, |
| | | insightful and inquisitive by nature. |
| 3 | Confident | Strong and Resilient towards the unknown. Ready to change as a process, |
| | | reflects confidence in move and display clarity in choices, generally is at peace |
| | | with self, demonstrates ample capability and willingness to trade off with the |
| | | fear of unknown ahead |
| 4 | Connected | Socially affable, Extroversion, enjoys being connected in social media and else |
| | | and driven by acceptance and recognition on scale |
| 5 | Experimentative | Abhors repetitiveness and actively explores unknowns, imaginative and |
| | | displays alternate thinking, considers the digital as a tool to achieve new |
| | | things, stays enthusiastic to bring in new ideas, and shows willing to adopt |
| | | changes. |
| 6 | Efficient | Demonstrates clarity & swiftness of actions and likes/dislikes, seeks |
| | | productivity in general and at workplace and lifestyle, considers digital to |
| | | generates & facilitate better decisions, undertakes productivity as a key |
| | | aspect in natural way of life |
| 7 | Epicurean | Driven by strong choices driven by hedonistic factors leading to content |
| | | driven consumption, invests time and interests towards self- |
| | | pleasure/entertainment. |

Table Signifying the Explanation of the 45 Behaviors which were Distilled into the 07 Traits in UCCCEEE Framework

TABLE 2.0
45 different behaviors leading towards the 07 personality traits of UCCCEEE framework

| SN | Keyword | Explanation of the behavioral parameter |
|---|---|---|
| 1 | Ease of Usage (EOU) | Ergonomics of the usage of the technology application as assessed by the |
| | | end user. The trade off as made by end user of learning curve/time in |
| | | hand for using the technologies. |
| 2 | The Fear of Missing | Fear Of Missing Out. The end user's concern that he/she may be left |
| | Out (FOMO), | behind in the associated social ecosystem an individual exists or operates |
| | | and may be looked down upon by others as outdated. |
| 3 | The Fear of Change | Fear associated by the end user towards suage of technologies citing that |
| | (FOC), | he/she may lose what is in hand to something which is not clearly |
| | | beneficial or carry a significant fear of unknowns. |
| 4 | Expression of having a | End users' representation of his/her profile to the world which may |
| | desire for an Alternate | consist of some hidden traits or otherwise insignificant traits. |
| | Profile | |
| 5 | Liking for efficiency in | Association of the end users with the usage of technologies for the direct |
| | lifestyle, | or indirect benefits in the area where the technologies are deployed. |
| 6 | Expression of | End users' ability to handle the technologies without any fear of usage or |
| | confidence, | disadvantage. |
| 7 | Seek out Popularity | Ability to leverage technologies to become popular in the wider masses. |
| | among the | |
| | surrounding | |
| | ecosystem and look | |
| | for self-differentiation, | |
| 8 | Representing through | Ability of creating a replica of end user personality traits over any virtual |
| | a Virtual Avatar | medium giving an end user a satisfaction of creating a virtual existence in |
| | beyond physical | social platform. |
| | boundaries, Being | |
| 9 | Being | End user intrinsic nature to explore more by being experimentative and |
| | Experimentative, - | without any fear of negative trade off while experimenting in lives. |
| 10 | Reflecting Age based | Demographic factor. It's assumed that younger person are born native to |
| | behaviors - faster | usage of the technologies. |
| | learning curve, | |
| 11 | Deeper liking for | Ability to align technologies to the personal like or dislike and thus find |
| | Personalization - | friendliness in the specific technologies by the end user. |
| | choices driven out of | |
| | personal likes and | |
| | dislikes | |
| 12 | Self-Gratification/ | End users' association of technologies with the want/desire of |
| | Entertainment - | entertainment on virtual platform like gaming, OTT or others. |
| | seeking diversified/ | |
| | personalized content, | |
| 13 | Staying Current/ | End user's intent and ability to gather adequate information on subjects |
| | Updated | of want, need and/or interests. |
| 14 | Aligning with Trends, | End user associating their usage of technologies with what is trending in |
| | | markets or socio-economic framework. Its inverse to the FOMO. |
| 15 | Seeking Happiness, | The degree of inherent happiness which is felt by the end user while |
| | | using technologies. Its inverse of the fear of change. |
| 16 | Staying Connected | The end users associated with technologies for their ability to |
| | with social ecosystem, | communicate with their social/personal or professional circles. |
| 17 | Enhanced liking for | End user's consideration that technologies are the levers of the next |
| | the Socialization, | frontier of the social transformation. |
| 18 | Driving through | Ability to expand conversations to unknown zones and individuals by |
| | Networking, | using the prowess of the technologies. |
| 19 | Having extra | End users' association to feeling secured in usage of the technologies - |
| | insistence of the | both in the inherent usage as well as by leveraging technologies to gain |
| | privacy and Security | security related advantages. |
| 20 | Display linguistic | Vernacular abilities across the usage of technologies. |
| | barriers | |
| 21 | Economic Affordability | Cost arbitrage of the using the technologies versus other alternatives. |
| 22 | Seeking reasonable | Ability to associate technologies to higher impact of the initiatives as |
| | impact, | attempted by the end users |
| 23 | Anxious to hear News, | End users' access to latest of the information over technologies enabled |
| | | platforms. |
| 24 | Creative and | Ability to make a quantum leap in innovation by leveraging technologies |
| | Innovating approach, | as compared to alternative methods. |
| 25 | Continuous Learning, | End users' ability to leverage the technologies towards the education of |
| | | the self. |
| 26 | Making significant | An individual desires to influence its ecosystem and make an impact in |
| | Influence | the surroundings |
| 27 | Staying always | Seeking relevance in the society and considering those factors which can |
| | Relevant | bring up the stature of the individual up in its area of existence |
| 28 | Deep Expressions | Ability to provide for a better method for expressing self. Especially for |
| | | introverts or who may like to camouflage under a given situation |
| 29 | Gathering and | Individual seeking higher state of awareness, awakening, understanding |
| | Retention of | or information gathering around the subject |
| | Knowledge, | |
| 30 | Logical Reasoning, | Individual seeking options to put up better articulation with support of |
| | | information and supporting in their defense or presentations. |
| 31 | Openness and | Openness of an individual to express beyond the normal realm of sphere |
| | Frankness, | of influence |
| 32 | Impact of Gender | Belief that genders biasness exists in choices made towards adoption of |
| | differentiation | emerging technologies and behavior guided by that - especially in |
| | | controlled societies |
| 33 | Quality of Education, | The education status of the individual and its impact on usage of the |
| | | emerging technologies. It is assumed that more learned a person is - |
| | | more its easier to adopt digital technologies. May not be true always |
| | | through. |
| 34 | Scale and time of | Conversational nature of an individual and time they spend on the same. |
| | Chatting | |
| 35 | Securing Privacy, | Innate need of individuals to keep privacy in their public disposition and |
| | | importance an individual attributes to this need. |
| 36 | Playful and | Basic nature of individuals to go out and explore more without much |
| | Adventurous, | stressed down by the boundary conditions or past experiences. |
| 37 | Nerdy and Technology | Emerging technologies across the world including the usage applications |
| | driven, | |
| 38 | Desire to display | Understanding of an individual about his/her surroundings |
| | degree of awareness, | |
| 39 | Economic disparity | Financial stability and economic conditions in which an individual exists |
| | through the family & | and aspires ahead |
| | individual income | |
| 40 | Reach and | Penetration of an individual into the supply chain of the products, |
| | Accessibility | services and associated information |
| 41 | Product offerings and | Product features and its influence on understanding of the individual/ |
| | Features | targeted respondent |
| 42 | Physical Touch feel | Ability to have a physical connect |
| 43 | Seeking Convenience | Ability to gather information and execute a transaction at ease |
| 44 | Outright Materialistic | Ambitions nursed by an individual and reflected through the usage of |
| | Ambitious | emerging technologies |
| 45 | Economic Models and | Financial advantages sought after by user |
| | Offers | |

The sample psychometric test given below is designed to ascertain the respondent behavior in each given situation. The sample test consists of 28 questions with 04 per category of the personality trait identified in the UCCCEEE framework. The questions below are sample and they are generated in the test paper through a random selection from a pool of questions. The respondents are expected to answer on a scale of 0 to 100 as given below.

The above description and illustrations should not be construed to restrict the scope of protection.

1. A system based on personality/behavioral traits which helps define & assess the digital quotient of an individual person or an organization, the said method comprising:
a psychometric test,
DQME scorecard,
DQME type indicators and
analysis of test results leading to profiling of respondents
Seven key personality traits derived through by 45 different behaviors across digital platform by an individual.
the said UCCCEEE framework of seven personality traits derived using natural language processing having the steps of:
a. word frequency cluster wise and using frequency as a differentiator selecting most frequently preferred content words by respondents while expressing behavior towards digital technologies adoption,
b. collocation in the text responses—relative rank ordering of these content words on conjugal appearances one or more times based on relevance score (RS) function, wherein, Function (RS)=(β/μ)(1),
where RS: relevance score
α=independent term frequency
β=associated term frequency (all combinations where the keyword was collocated with other keywords and in text responses)
μ=α+β=Total content words collected in the bag of words for the cluster.
Relevance score method gives the moderation towards identifying the uniqueness of the above said content word. This enables identification of the priority ranking of the content words and wherever more than one cluster were involved, the RS were considered as aggregated values for many clusters.
a. Natural language processing comprising the term frequency formula:
$${TF}_{i , j} = \frac{{n}_{i , j}}{{\sum }_{k} {n}_{i , j}}$$
where n=number of times the word has occurred and is divided by the number of terms in a document.
and inverse frequency formula:
$${idf}_{j} = log [ \frac{n}{{df}_{i}} ]$$
where n represents the total number of documents and is divided by the number of documents containing the given term
the term Frequency and Inverse frequency relationship indicated by:
$${w}_{i , j} = {tf}_{i , j} \times log ( \frac{n}{{df}_{i}} )$$
$$\begin{matrix}{tf}_{ij} = number of occurrences of i in j \\ {df}_{i} = number of documents containing i \\ N = total number of documents\end{matrix}$$
wherein it results in assessment of digital quotient of an individual or an organization which helps in analyzing the correlation between an individual person and the surrounding digital ecosystem. The said DQME Type Indicators to represent an individual entity/person's Digital Quotient as measured through said psychometric testing method,
the result of the psychometric testing score resulting as “DQME Rank Order Score” of an individual.
the explanation of the behaviors associated with the DQME score indicating the “DQME Type Indicators” of an individual.

2. The system as claimed in claim 1, wherein, the said framework of these 07 personality traits is referred to OR re-called as “Shandilya UCCCEEE framework” wherever third party uses this framework for any authorized usage.

3. The system as claimed as claim 1, wherein, the said seven human personality traits identified as UCCCEEE are Updated, Curious, Confident, Connected, Efficient, Experimentative and Epicurean that are derivative and representative coverage of over 45 different human behaviors which an individual displays while dealing with various digital/information technologies.

4. The system as claimed in claim 1, wherein, DQME Rank Order Score is a direct sum-total measure of respondent's digital quotient which represents a collective value of response to the situational questions under all seven personality trait categories in the psychometric test.

5. The system as claimed in claim 1, wherein, the measuring is based on a range scale of 1 to 100 such that, higher the score, more it is conducive to the positive response to a stimulus at a very high level and is considered high-high positive correlation.

6. The system as claimed in claim 1 wherein, the UCCCEEE framework with range score is considered:
a. UPDATED (between range of 0 to 100 and point of inflexion as 50)
b. CONFIDENT (between range of 0 to 100 and point of inflexion as 50)
c. CONNECTED (between range of 0 to 100 and point of inflexion as 50)
d. CURIOUS (between range of 0 to 100 and point of inflexion as 50)
e. EFFICIENT (between range of 0 to 100 and point of inflexion as 50)
f. EPICUREAN (between range of 0 to 100 and point of inflexion as 50)
g. EXPERIMENTATIVE (between range of 0 to 100 and point of inflexion as 50)

7. The system as claimed in claim 1, wherein, the respondent answers situational questions per category under each of the seven trait categories and weighted average score per trait categories are calculated on a range score of 1 to 100.

8. The system as claimed in claim 1, wherein, when the trait wise system results are represented in binary mode—the weighted average score between 0 to 50 is represented as “0” and score between 51 to 100 is considered as “1”.

9. The system as claimed in claim 1, wherein, representation of the DQME Type Indicator Score is considered as sequence of “Binary Scores” across the seven traits in sequence.

10. The system as claimed in claim 1, wherein, term frequency and inverse frequency relationship extract keywords that are most frequently used by respondents across all clusters.

11. A method of psychometric test comprising the system as claimed in claim 1.
